(19) United States
(12) Patent Application Publication
(10) Pub. No.: US 2016/0296267 A1
(43) Pub. Date: Oct. 13, 2016

(54) SYSTEM AND APPARATUS FOR PERFORMING TRANSFORMINAL THERAPY

(71) Applicant: VANDERBILT UNIVERSITY, Nashville, TN (US)

(72) Inventors: Joseph Neimat, Nashville, TN (US); Eric J. Barth, Nashville, TN (US); Robert J. Webster, Nashville, TN (US); David B. Comber, Nashville, TN (US)

(21) Appl. No.: 15/037,074

(22) PCT Filed: Nov. 17, 2014
(86) PCT No.: PCT/US2014/065898

§ 371 Date: May 17, 2016

Related U.S. Application Data
(60) Provisional application No. 61/905,534, filed on Nov. 18, 2013.

(51) Int. Cl. A61B 18/00; A61B 17/34; A61B 34/00; A61B 34/30

(52) U.S. Cl. CPC A61B 18/00; A61B 34/30; A61B 17/3421; A61B 34/70; A61B 2018/00184; A61B 2034/301; A61B 2034/302; A61B 2017/003

(57) ABSTRACT

A system and apparatus for performing transforaminal therapy utilizes a cannula positioned in the foramen ovale and a probe that is operable through an actuator to access the brain via the cannula. According to one aspect, the actuator can be a manual mechanical actuator. According to another aspect, the actuator can be a robotic actuator. According to a further aspect, the actuator can be adapted for use in an imaging environment, such as a magnetic resonance imaging (MRI) system.

### RELATED APPLICATIONS

This application claims the benefit of U.S. Provisional Application No. 61/905,534, filed Nov. 18, 2013, the disclosure of which is hereby incorporated herein by reference in its entirety.

### GOVERNMENT FUNDING

This invention was made with government support under Grant No. 0540834 awarded by The National Science Foundation, Center for Compact & Efficient Fluid Power. The United States government has certain rights to the invention.

### TECHNICAL FIELD

The present invention relates to a system, method, and apparatus for performing transforaminal therapy. In one particular aspect, the invention relates to a system, method, and apparatus for performing a neurosurgical procedure utilizing a cannula positioned in the foramen ovale and an active/steerable robotic probe that accesses the brain via the cannula. According to one aspect, the robot can be one adapted for use in a magnetically-sensitive environment, such as that of a magnetic resonance imaging (MRI) system.

### BACKGROUND OF THE INVENTION

Surgical resections for epilepsy and tumor resections are routinely performed through a craniotomy requiring a surgery of several hours, a post-operative ICU stay and significant potential morbidity and discomfort. Percutaneous techniques have been previously developed using stereotactic frames, but these also require surgery to drill the skull and enter the brain. The mesial structures of the temporal lobe are the most commons location of epileptogenic foci. These structures lie directly adjacent and lateral to the foramen ovale, a small opening in the base of the skull. The foramen ovale is routinely accessed via needle to advance electrodes that record activity from the medial edge of the hippocampus.

MRI provides good contrast between the different soft tissues of the body, which makes it especially useful in imaging the brain, muscles, the heart, and cancers compared with other medical imaging techniques such as computed tomography (CT) or X-rays. Unlike CT scans or traditional X-rays, MRI uses no ionizing radiation, so prolonged exposure in an MRI environment poses no danger to the patient or physician. The MRI equipment therefore can be ideal for use in monitoring and visualization in various medical procedures, and. uniquely offers capabilities such as thermal dosimetry by MR thermometry. The very high strength of the magnetic field does, however, require that ferromagnetic and other objects not compatible with an MRI operating environment not be present during the MRI monitored procedure. Moreover, the presence of non-MRI compatible materials and objects can cause inaccuracies or errors in the MRI imaging, and the radiofrequency signals produced by the scanner can negatively affect performance of robotic devices inside the scanner.

Active or steerable cannulas or probes are robotic devices that can be used to deliver various medical treatments or procedures, such as ablations (acoustic, thermal, laser), biopsies, deep brain stimulation, and electrode placement. Active probes include a plurality of concentric or nested tubes which may each have preformed curvatures and/or predefined flexibilities. The translation and/or angular orientation (rotation) of each tube may be controlled individually such that the tubes can telescope and rotate to move the tip of the cannula to a desired orientation and along a desired path. The tip of the cannula may he adapted to carry a tool such as biopsy tools, forceps, scalpels, ablation electrodes/transducers, stimulation electrodes, or cameras.

In certain procedures, such as neurosurgical procedures, precise control of the active cannula or probe is of the utmost importance. This precise control can be facilitated via an active/steerable probe robot. In performing these precision procedures, MRI imaging can be very helpful in providing guidance and feedback to the surgeon performing the procedure. In doing so, however, the robot and cannula/probe must have a construction that is compatible with use in an MRI environment.

### BRIEF DESCRIPTION OF THE DRAWINGS

For a better understanding of the invention, reference may be made to the accompanying drawings.

FIG. 1 is a perspective view illustrating a patient undergoing treatment according to an embodiment of the invention.

FIG. 2 is a side view illustrating the treatment of FIG. 1.

FIG. 3 is a top view illustrating the treatment of FIG. 1.

FIG. 4 is a side view illustrating the treatment of FIG. 1 in relation to the patient's skeletal and neurological structures.

FIG. 5 is a superior view illustrating the treatment of FIG. 1 within the cranial structure of the patient.

FIGS. 6A and 6B are perspective views of an apparatus that forms a portion of a system for performing the treatment illustrated in FIGS. 1-5.

FIGS. 7A and 7B illustrate a potion of the apparatus of FIG. 5.

FIGS. 8-10 are block diagrams illustrating methods performed by the system and apparatus of FIGS. 6A-7B to apply the treatment of FIGS. 1-5.

### DESCRIPTION OF EMBODIMENTS

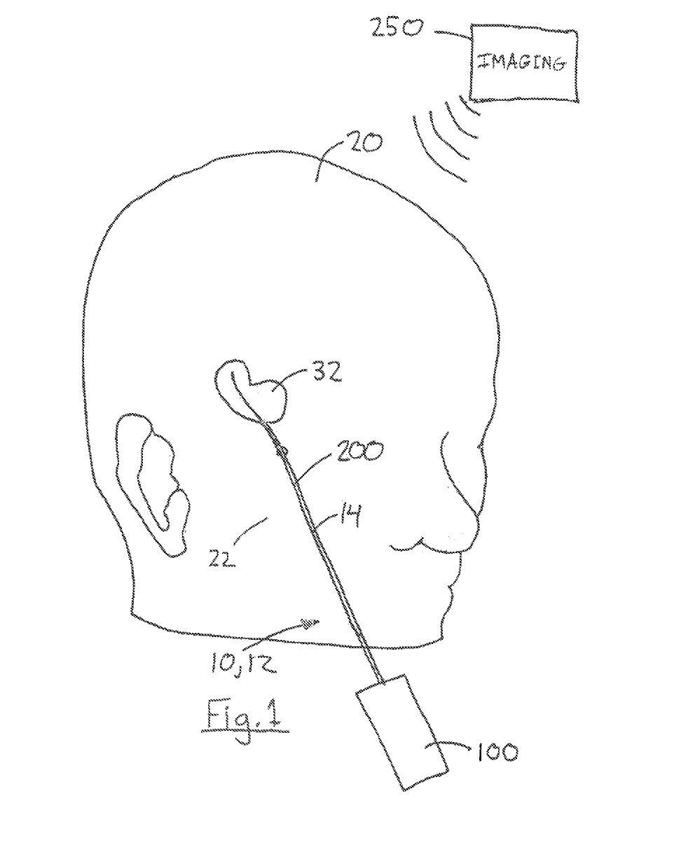

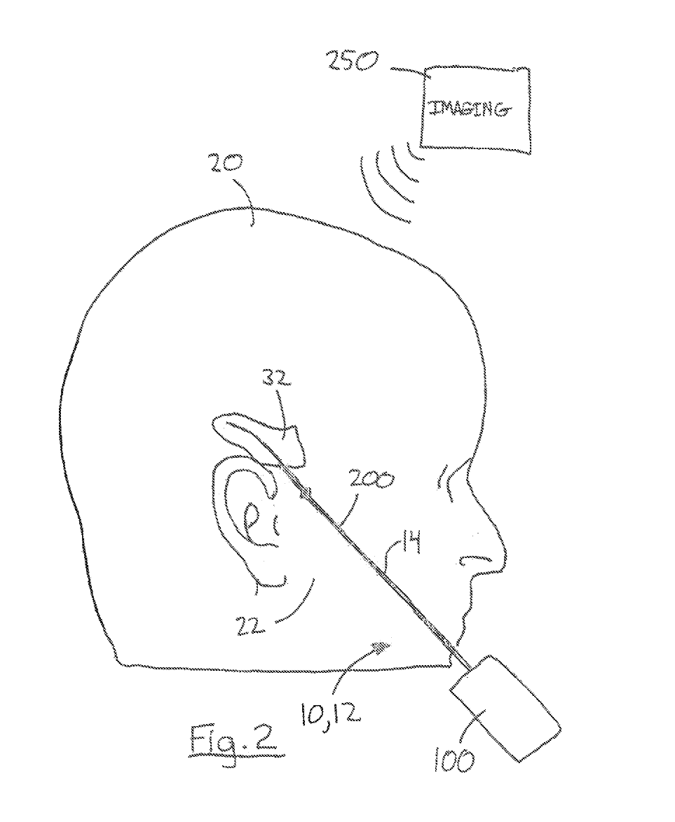

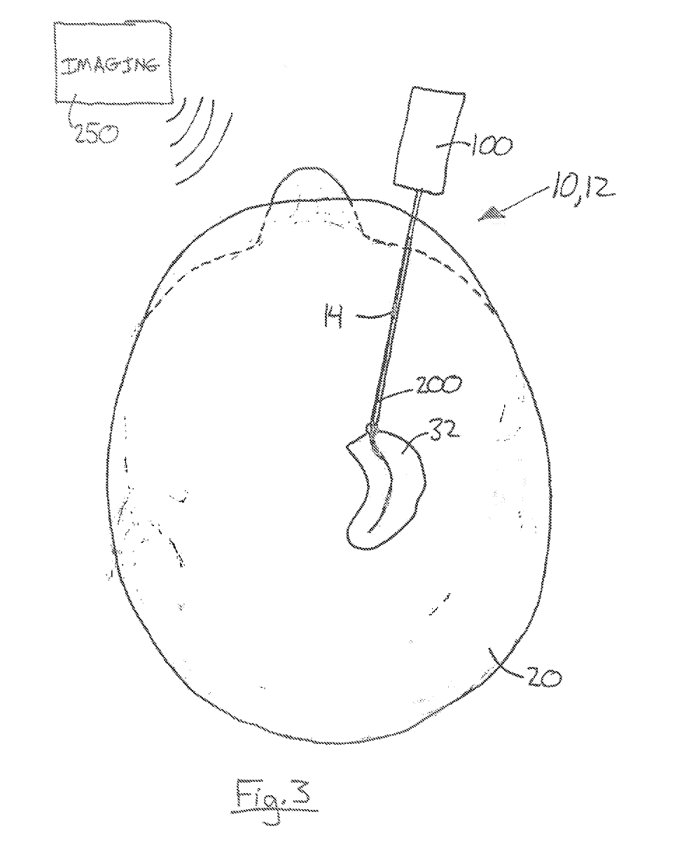

According to the invention, a system, method, and apparatus is utilized to perform transforaminal therapy. Referring to FIGS. 1-5, according to one example embodiment, a system 10 includes an apparatus 12 for performing a neurosurgical procedure on a patient 20. According to the procedure, the patient's brain 28 is accessed through the foramen ovale 24—one of several holes, or foramina, that transmit nerves through sphenoid bone of the skull 26. The patient 20 is fit with a cannula 14 that is inserted through the cheek 22 and guided through the foramen ovale 24 on either side (left or right) of the skull 26 to access the brain 28 in a known manner. This can be done, for example, using a standard cannulation needle under fluoroscopic guidance.

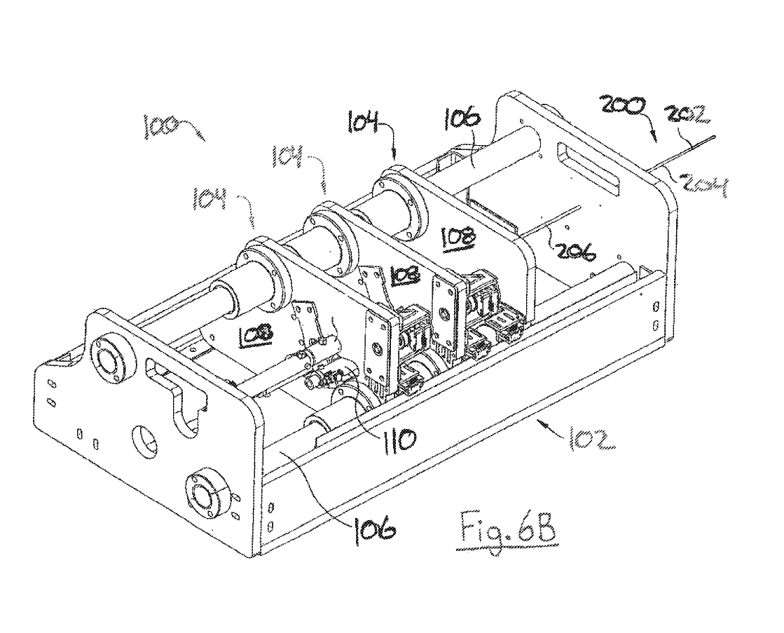

Once the foramen ovale 24 is cannulated, a surgical instrument, such as a probe 200 can be actuated to access and treat the brain 28. Referring to FIGS. 7A and 7B, the probe 200 is a concentric tube probe. The probe 200 of this example embodiment is a three tube probe that includes innermost, middle, and outermost concentrically nested tubes 202, 204, and 206, respectively. The probe 200 could include a greater number or fewer tubes. An end effector or tool 208 is located at the distal end of the innermost tube 202. The tool 208 can, for example, be a biopsy tool, forceps, scalpel, ablation electrode/transducer, stimulation electrode, or camera. As an example, the probe 200 can be similar or identical in design and function to the probe described in U.S. patent application Ser. No. 12/084,979, now issued U.S. Pat. No. 8,152,756, the disclosure of which is hereby incorporated by reference in its entirety.

As shown in FIGS. 7A and 7B, the tubes 202, 204, and 206 may collectively define and extend along a longitudinal tube axis 210. The tubes 202, 204, and 206 can have different configurations and material constructions. For example, the outermost tube 206 can be a rigid (e.g., titanium) tube, and the middle tube 204 and innermost tube 202 can be nitinol tubes. These nitinol tubes can be pre-curved to allow for steering the probe 200 through translational movement (i.e., movement along the axis 210) and rotational movement (i.e., movement about the axis 210) of the respective tubes, either individually or in combination. Although referred to herein as a “tube,” the innermost tube 206 is not necessarily hollow and could, for example, be a solid wire.

The probe 200 can have several degrees of freedom. In the example embodiment of FIGS. 7A and 7B, the three tube probe 200 can have five degrees of freedom. In this example configuration, the outermost tube 206 can be configured to permit translational movement along the axis 210. The middle tube 204 and innermost tube 202 can be configured to permit translational movement along the axis 210 and rotational movement about the axis. All of these degrees of freedom are available independently of each other and can be performed sequentially or simultaneously. These independently moveable degrees of freedom in combination with the pre-curvature of the tubes allows for steering the probe 200 along a desired path and to a desired site. Through the addition or removal of tubes, the probe 200 could be configured to provide additional degrees of freedom or fewer degrees of freedom, respectively.

Referring generally to FIGS. 1-5, the probe 200 can be actuated in a variety of manners, including robotic actuation and manual mechanical actuation, or a combination of robotic and manual actuation, in order to position the probe at the desired location in the patient 20. The actuator for providing this robotic and/or manual actuation is illustrated schematically at 100 in FIGS. 1-5. The actuator 100 is illustrated schematically in FIGS. 1-5, and this illustration is not meant to indicate its relative size.

The actuator 100 is configured to impart translational and/or rotational movement to some or all of the tubes 202, 204, 206 in order to operate the probe 200 with some or all of its multiple degrees of freedom. All degrees of freedom of the probe 200 are not necessarily afforded by the actuator 100 alone. Some degrees of freedom of the probe 200 can be afforded through the manual manipulation of the physical position and/or orientation of the entire apparatus 12 itself. Translational movement of any particular tube or tubes can be achieved through manual linear movement of the entire apparatus 12. Similarly, rotational movement of any particular tube or tubes can be achieved through manual rotational manipulation of the entire apparatus 12. These movements can be achieved through the use of a mounting structure to which the apparatus 12 is mounted, such as an orthogonal frame. The mounting structure can assist the surgeon in maneuvering the apparatus 12 and can be locked to fix the position of the apparatus. Once the manual operation is complete, the position of the apparatus 12 can be fixed relative to the patient via the mounting structure.

For instance, in one particular configuration, the probe 200 can be configured with 4 degrees of freedom: two translational and two rotational. In this configuration, the outermost tube 206 can be fixed and not configured for translational or rotational movement via the actuator. The middle tube 204 and the innermost tube 202 are configured for translational and rotational movement via the actuator 100. In this configuration, initial placement of the probe 200 is performed manually by the surgeon. During this initial placement, the middle tube 204 and innermost tube 202 can be retracted into the outermost tube 206. The surgeon manually positions the apparatus 12 with the middle and innermost tubes 204 and 202 retracted into the outermost tube 206 in order to perform initial positioning of the probe 200. In this example configuration, the surgeon can control this initial probe positioning manually without any assistance from the actuator 100. Once the position of the apparatus 12 is locked relative to the patient, the actuator 100 can take over further operation of the probe 200.

Those skilled in the art will appreciate that, through the operation described above, the apparatus 12 is configured to provide multiple degrees of freedom of the probe 200 through the actuator 100 or through manual positioning of the apparatus in any desired combination. Thus, the apparatus 12 could be configured for course control of the probe 200 through manual operation and for fine control through operation via the actuator 100. The actuator 12 can be configured so that this fine control can be executed with sub-millimeter precision.

The actuator 100 can be a robotic or a manually actuated mechanism. Regardless of the configuration of the actuator 100, operation of the probe 200 can be performed with or without the aid of an imaging or visualization system, such as an MRI, fluoroscopy, CT scan, or ultrasound, which is indicated. generally at 250. In an MRI-compatible configuration, the actuator 100 is a nonmagnetic device that includes nonmagnetic manual and/or robotic components.

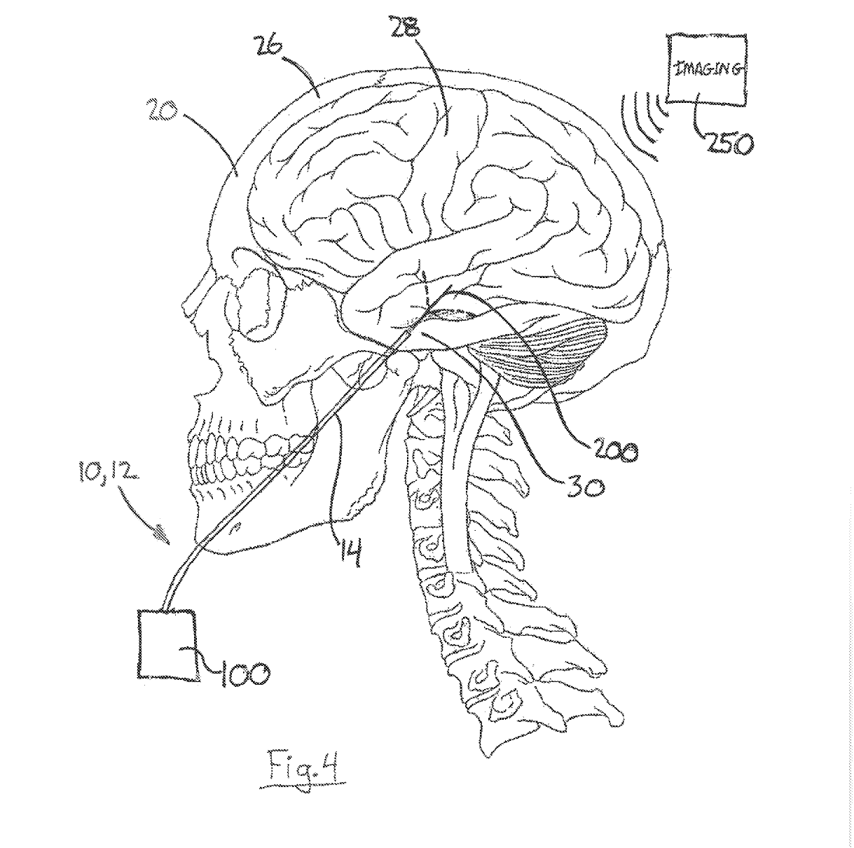

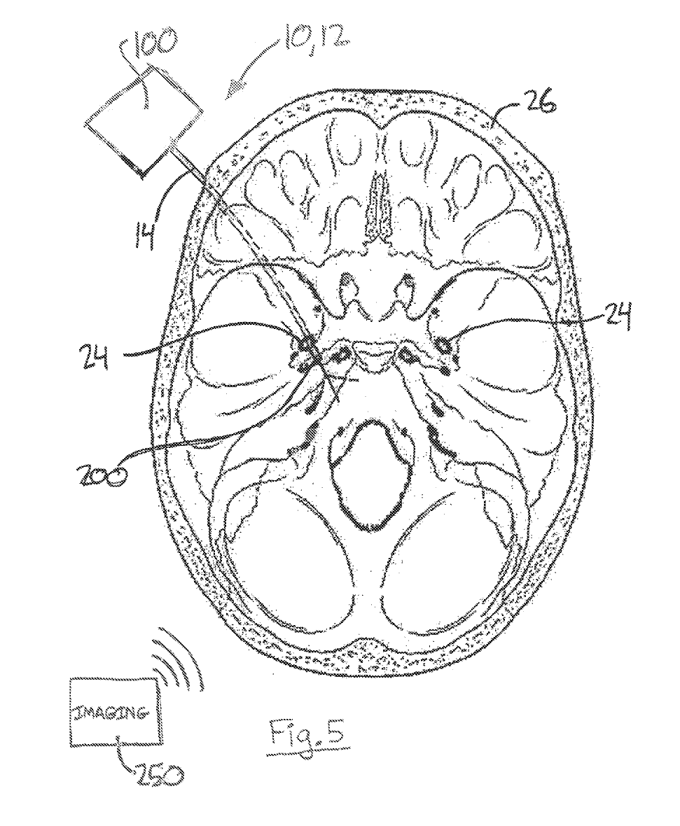

Under robotic control, an actuator 100 in the form of a robot actuates (e.g., steers, operates, manipulates) the probe 200 in a desired manner. For example, the robot 100 can be controlled to steer the probe 200 along a desired path to a desired location in the brain 28, as indicated generally by the dashed lines in FIGS. 4 and 5. Once at the desired location, the probe 200 can be operated to perform the desired surgical operation (e.g., ablation) or to apply the desired therapy (e.g., stimulation).

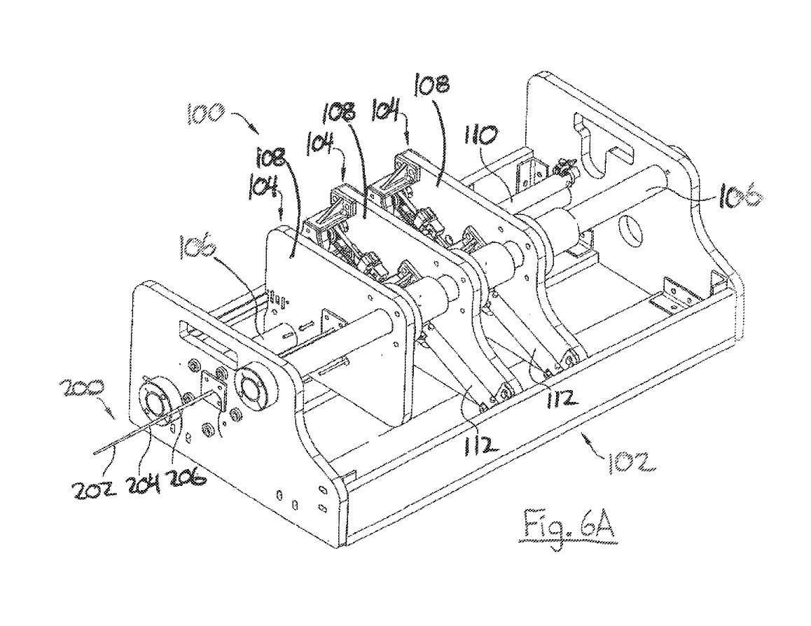

A multiple degree of freedom robotic device 100 that can be used to perform the transforaminal procedure in an MRI environment is illustrated in FIGS. 6A and 6B. The robot 100 can, for example, be a robot that is similar or identical in design and function to the robot described in U.S. patent application Ser. No. 13/679,512 (see U.S. publication US 2013/0123802 A1), the disclosure of which is hereby incorporated by reference in its entirety.

The robot 100 is constructed and configured to produce some or all of the degrees of freedom of the tubes 202, 204, 206 referred to above. Referring to FIGS. 6A and 6B, the robot 100 includes a rigid box frame 102 that supports modules 104 associated with a corresponding one of the tubes 202, 204, 206. The modules 104 translate along guiding rods 106. Each module 104 includes a base in the form of a plate 108 that translates via bearings along the guide rods 106.

Each module 104 includes a translational actuator 110 for translating the associated plate 108 and its associated tube along the guide rods 106 and along the axis 210. Each module 104 can also include a rotational actuator 112 for rotating its associated tube about the axis 210. Because the outermost tube 206 may not be adapted for rotation, the module associated with the outermost tube 206 may not include a rotational actuator, or that actuator may be disabled or simply not used. In an MRI compatible configuration of the robot 100, these actuators 110 and 112 can be constructed of MRI compatible materials and may be operated, for example, pneumatically (e.g., via pneumatic stepper motors). Alternatively, the use of piezoelectric actuators can also be implemented in an MRI compatible manner.

Additionally, in a scenario where alternative imaging systems are utilized MRI compatibility is not an issue in the construction of the robot 100. For example, the system 10 and apparatus 12 may be employed under fluoroscopy or other imaging methods like CT or ultrasound. In this instance, the actuators 110 and 112 can have any desired configuration and material construction that is consistent with these imaging techniques.

Linear position sensing of the modules 104 can be accomplished via one or more optical linear encoders, and rotational position sensing can be accomplished via one or more optical rotary encoders monitoring the actuators 112. Alternatively, stepper motors can be implemented which, due to their operational characteristics, can provide inherent positional awareness. The robot 100 can thus be controlled in a known manner to cause translational and rotational actuation of the tubes 202, 204, 206 in order to produce movement of the tool/ablation element 208 along the desired path to the desired location. It will therefore be appreciated that, for a patient that has a transforaminal cannula 14 (see, FIGS. 1-5) positioned through the foramen ovale 22, the robot 100 can access the brain 20 and can be used to steer the probe 200 to the desired location in the brain. Once at the desired location, the probe 200 can be actuated to perform the desired surgical operation (e.g., ablation) or to apply the desired therapy (e.g., stimulation).

Under manual mechanical actuation, the actuator 100 comprises one or more manually operated machines or mechanisms that are used to operate (e.g., steer, manipulate, actuate) the probe 200 in order to produce the desired movements of the probe. Through the mechanical actuator 100, the probe 200 can be manually operated to direct the probe 200 along a desired path to a desired location in the brain 28, as indicated generally by the dashed lines in FIGS. 4 and 5. Once at the desired location, the probe 200 can be actuated to perform the desired surgical operation (e.g., ablation) or to apply the desired therapy (e.g., stimulation).

The mechanical actuator 100 can have a variety of configurations. The mechanical actuator 100 can be configured exclusively for manual operation or can be fit for a combination of mechanical and assisted (e.g., servo assisted) operation. For example, the mechanical actuator 100 can have a configuration that is essentially the same as the robotic actuator of FIGS. 6A and 6B, except that the modules for imparting translational and rotational movement of the tubes 202, 204, 206 would be actuated manually (e.g., through knobs, levers, thumb wheels, etc.) to produce the desired movement.

As another example, the mechanical actuator 100 can be an actuator that is similar or identical in design and function to any of the configurations described in U.S. patent application Ser. No. 12/921,575 (see U.S. publication US 2011/0015490 A1), the disclosure of which is hereby incorporated by reference in its entirety. In this instance, the nested tubes 202, 204, 206 can be mounted to respective blocks that, in turn, are mounted to tracks in a manner such that the blocks can slide linearly relative to each other and thereby produce translational movement of the tubes along the tracks and along the axis 210. Through this linear motion, the tubes 202, 204, 206 can be moved individually relative to each other, can be telescoped, and the probe 200 as a whole can be advanced. The blocks can also be configured to allow independent manual rotation of the tubes 202, 204, 206 and thereby provide rotational movement of the tubes relative to each other about the axis 210. Through this configuration, the mechanical actuator 100 can provide some or all of the degrees of freedom of the probe 200.

The apparatus 12 can be used, manually, robotically, or a combination of manually and robotically, to perform a variety of procedures. For example, the apparatus 12 can be used for the ablation (e.g., ultrasound, laser or RF ablation) of structures and lesions in the brain 28. For instance, the apparatus 12 can be used to ablate lesions or tumors of the temporal lobe 30 (including the uncus, amygdala, hippocampus 32 and parahippocampal gyrus for the treatment of epilepsy). Tumors and lesions elsewhere in the brain, such as in the deep brain structures or other lobes of the brain, can also be accessed and treated in this manner. Deep brain stimulation and electrode placement can also be achieved in this manner.

These procedures can be performed using the transforaminal approach of the invention using the apparatus 12 without any incision and while avoiding the need to drill or otherwise form an opening in the skull 26. This minimally invasive procedure can be performed outside the operating room in an MRI scanner or under other imaging techniques. The procedure can be much faster than conventional surgeries and can have significantly lower morbidity and patient discomfort.

One particular area in which this transforaminal approach can be especially beneficial is in the treatment of temporal lobe epilepsy. Under this approach, the probe 200 can be operated to carry an ablation element 208 to ablate the hippocampus to help treat this condition. Through this system 10, a complete ablation of the hippocampus 32, with the potential to cure epilepsy, could be performed while enjoying all of the benefits of this minimally invasive approach.

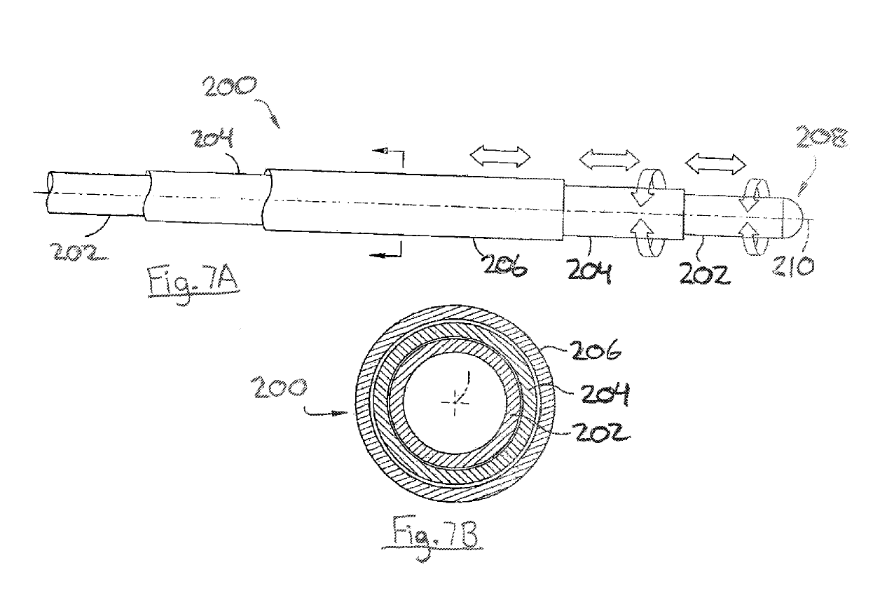

From the above, those skilled in the art will appreciate that, according to another aspect of the invention, the disclosed system 10 and apparatus 12 are used to perform a method for applying therapy to the brain 28. Referring to FIG. 8, the method 120 includes the step 122 of cannulating the foramen ovale of a patient. At step 124, a probe accesses the brain via insertion through the transforaminal cannula. At step 126, the probe is steered to a site in the patient's brain. This steering can be achieved manually, robotically, or a combination of manually and robotically. At step 128, therapy is applied to the brain at the site.

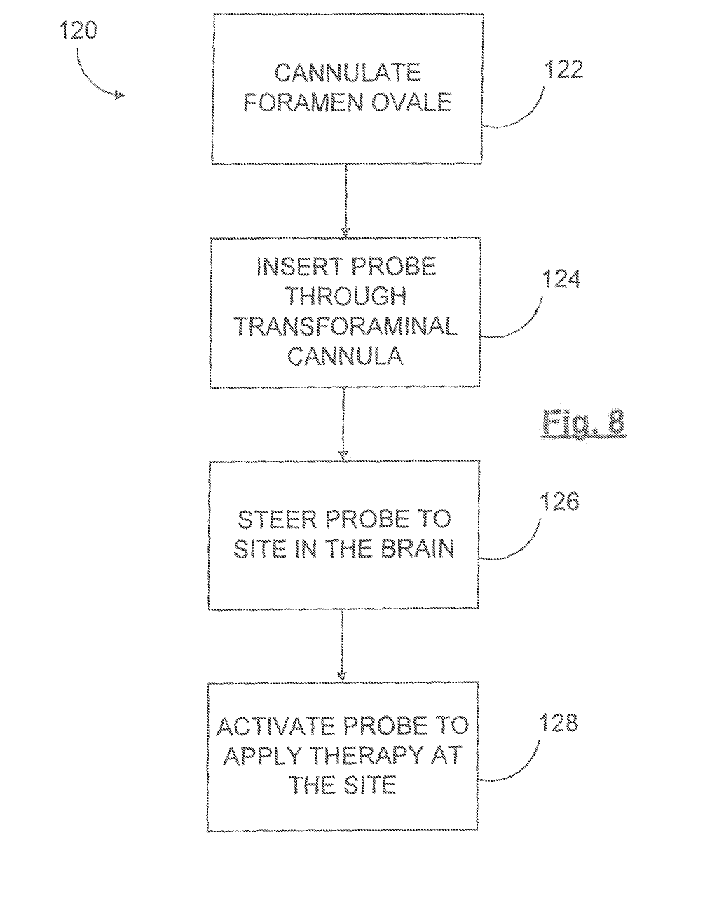

Referring to FIG. 9, according to another aspect of the invention, the step 124 of steering the probe includes the step 130 of guiding the probe remotely, and the step 132 of using MRI visualization to monitor the progress of the probe in the patient. These steps 130 and 132 can be repeated many times and in any order. For example, one skilled in the art can appreciate the desirability of establishing MRI visualization prior to advancing or otherwise manipulating the probe.

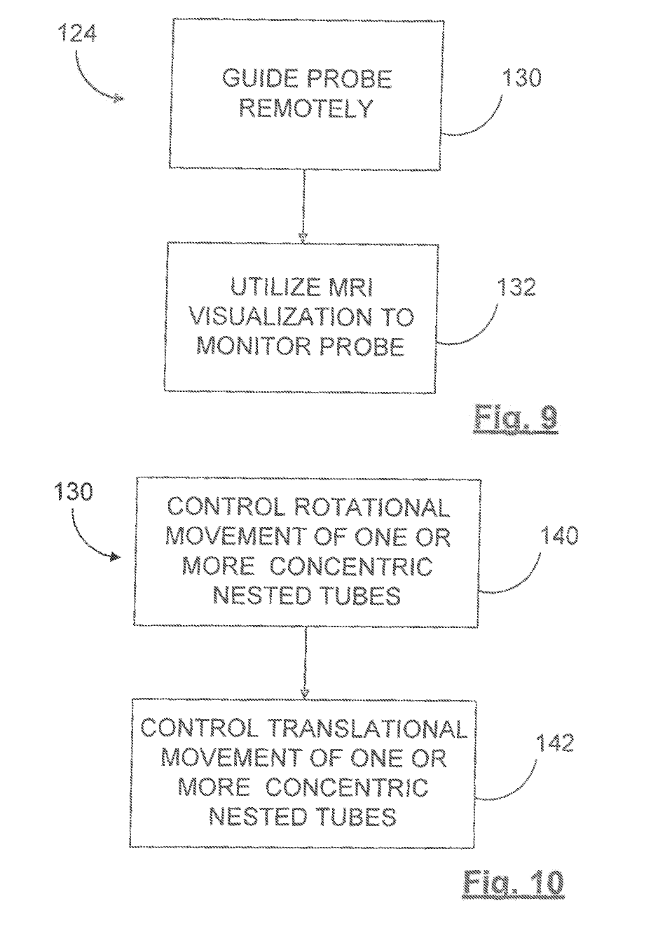

Referring to FIG. 10, according to another aspect, the step 130 of guiding the probe comprises the step 140 of controlling the rotational movement of one or more concentrically nested tubes, and the step 142 of controlling the translational movement of the one or more concentrically nested tubes. Again, these steps 140 and 142 can be repeated many times and in any order. As such, the order in which the steps 140 and 142 are performed is not important. One skilled in the art can easily perceive that, in a procedure that includes many tens or hundreds of individual probe movements, the order in which certain subsets of steps are performed is relative. Controlling the rotational and translational movement of the tubes can be achieved manually, robotically, or a combination of manually and robotically.

From the above, those skilled in the art will appreciate that the system 10, apparatus 12, and method 120 of the invention affords a novel neurosurgical approach for accessing the brain via the foramen ovale. According to one aspect of the invention, this transforaminal access can be achieved, at least in part, robotically. By “robotic” or “robotically,” it is meant to describe the operation—movement, manipulation, steering, and actuation—of the robotic components (e.g., the probe 200) facilitated by the robot 100. Control of the robot 100 to operate the probe 200 can be achieved in different manners. For example, the robot 100 could be controlled automatically via computer control whereby a computer is programmed to control the robot in order to operate the probe 200 to perform the desired surgical operation. As another example, the robot 100 could be controlled manually, e.g., through a remote or local control interface such as a joystick controller or other handheld controller such as one similar to the familiar videogame-style controllers, to operate the robot in order to direct the probe 200 to perform the desired surgical operation. Additionally, a hybrid approach could be employed in which the robot 100 could be controlled through a combination of computer and manual controls to operate the probe 200 to perform the desired surgical operation.

While aspects of the present invention have been particularly shown and described with reference to the preferred embodiment above, it will be understood by those of ordinary skill in the art that various additional embodiments may be contemplated without departing from the spirit and scope of the present invention. Other aspects, objects, and advantages of the present invention can be obtained from a study of the drawings, the disclosure, and the appended claims.

Having described the invention, we claim:

1. A method for applying therapy to the brain of a patient, comprising:
cannulating the foramen ovale of the patient;
inserting a probe though the transforaminal cannula;
steering the probe to a site in the brain; and
activating the probe to apply therapy to the brain at the site.

2. The method recited in claim 1, wherein the step of steering the probe comprises guiding the probe remotely and using MRI visualization to monitor the progress of the probe.

3. The method recited in claim 2, wherein the step of guiding the probe comprises controlling the translational movement of concentrically nested tubes, and controlling the rotational movement of the concentrically nested tubes.

4. The method recited in claim 1, wherein the step of steering the probe comprises using a robotic actuator to steer the probe.

5. The method recited in claim 1, wherein the step of steering the probe comprises using a manual mechanical actuator to steer the probe.

6. The method recited in claim 1, wherein at least one of the concentrically nested tubes are pre-curved.

7. The method recited in claim 1, wherein the step of steering the probe comprises the step of providing an MRI compatible actuator.

8. The method recited in claim 1, wherein the step of applying therapy comprises performing an ablation of at least one of structures and lesions of the brain.

9. The method recited in claim 8, wherein the step of performing an ablation comprises performing an ablation of the temporal lobe.

10. The method recited in claim 8, wherein the step of performing an ablation comprises performing an ablation of the hippocampus to treat epilepsy.

11. The method recited in claim 1, wherein the step of applying therapy comprises performing deep brain electrode placement.

12. A system for applying therapy to the brain of a patient, comprising:
a cannula for cannulating the foramen ovale of the patient;
a probe insertable through the cannula to probe the brain; and
an actuator for steering the probe in the brain.

13. The system recited in claim 12, wherein the probe comprises a concentric nested tube probe.

14. The system recited in claim 13, wherein the nested tube probe comprises at least one pre-curved tube.

15. The system recited in claim 13, wherein the nested tube probe has an axis, the actuator being operable to translate the at least one pre-curved tube along the axis and to rotate the at least one pre-curved tube about the axis.

16. The system recited in claim 12, wherein the actuator comprises a robotic actuator.

17. The system recited in claim wherein the actuator comprises a manual mechanical actuator.

18. The system recited in claim 12, wherein the probe comprises an end effector for applying the therapy.

19. The system recited in claim 18, wherein the end effector comprises an ablation element.

20. An apparatus for applying therapy to the brain of a patient, comprising a concentric nested tube probe that is actuatable and that is adapted to access the brain through a cannula inserted in the foramen ovale of the patient.
